(19) United States
(12) Patent Application Publication
(10) Pub. No.: US 2025/0322512 A1
(43) Pub. Date: Oct. 16, 2025

(54) PARAMETER ESTIMATION METHOD FOR COMPARTMENT MODEL BASED ON PHYSICS-INFORMED NEURAL NETWORKS

(71) Applicant: ZHEJIANG UNIVERSITY, Hangzhou (CN)

(72) Inventors: HUAFENG LIU, HANGZHOU (CN); HENGJIA RAN, HANGZHOU (CN)

(21) Appl. No.: 18/932,929

(22) Filed: Oct. 31, 2024

(30) Foreign Application Priority Data
Apr. 10, 2024 (CN) 2024104271877

(51) Int. Cl. G06T 7/00; G06T 7/11

(52) U.S. Cl. CPC G06T 7/0012; G06T 7/11; G06T 2207/20081; G06T 2207/20084; G06T 2207/30024; G06T 2207/30101; G06T 2207/30204

(57) ABSTRACT

The present invention is a parameter estimation method for compartment model based on physics-informed neural networks. Starting from a physical model, the method extracts information from an AIF and a small amount of measurement data to obtain kinetic parameters, thereby greatly improving the scanning efficiency of a measuring instrument, and reducing occurrence of inaccurate estimation results due to patient movement. In addition, the present invention has the robustness to AIF noise and measurement data noise, and can flexibly arrange the time of data acquisition, reduce an error of inaccurate estimation caused by long time 10 acquisition and the patient movement, and improve the efficiency of data acquisition of the instrument. Experimental results show that the present invention is more stable and has less errors. Meanwhile, the present invention does not require the setup of training datasets, and is superior to an end-to-end supervised reconstruction method U-net network with fewer samples.

### FIELD OF TECHNOLOGY

The present invention belongs to the technical field of kinetic parameter imaging, and in particular relates to a parameter estimation method for compartment model based on physics-informed neural networks.

### BACKGROUND TECHNOLOGY

Compartment models are widely used in many branches of life science, such as pharmacokinetics in pharmacology, metabolic system researches, medical imaging analysis and other fields. A basic method of this modeling manner is to analyze a system by breaking it down into a limited number of regions (called compartments or states), which interact with each other through exchange of substances. A compartment system is modeled primarily in a continuously deterministic manner by a set of ordinary differential equations, each describing a change rate in the number of substances in a particular compartment, and the change rate is determined by the physicochemical laws that govern the exchange of substance among the compartments, such as diffusion, temperature, biochemical reactions.

In addition to difficulty in forward modeling of the compartment model, another problem of the compartment system is to deal with an inverse problem, that is, parameter estimation. Many traditional methods have been widely used in estimation of kinetic parameters, which can be classified into two types, namely nonlinear estimation and linear estimation. The former comprises methods of nonlinear least square (NLLS), iteratively weighted NNLS (IRWNLLS) and nonlinear ridge regression (NLRR). The latter comprises methods of linear least square

(LLS), total least square (TLS), and a basis function (BF). However, when there is high noise, some solutions may be unstable at a voxel level and in a region of interest (ROI). In addition, traditional methods often need to know full measurement data for accurate parameter estimation, and longtime data acquisition may lead to an inaccurate result due to object movement, while also reducing the efficiency of the instrument.

With development of deep learning, several new methods have been used to estimate kinetic parameters, and in the field of positron emission tomography (PET), literature [Hong J, Brendel M, Erlandsson K, et al. Forecasting the pharmacokinetics with limited early frames in dynamic brain pet imaging using neural ordinary differential equation[J]. IEEE Transactions on Radiation and Plasma Medical Sciences, 2023] proposes to utilize a neural ODE method to predict the full frame PET images and reconstruct parameter images in data-driven manner; and literature [Liang G, Zhou J, Chen Z, et al. Combining deep learning with a kinetic model to predict dynamic PET images and generate parametric images[J]. EJNMMI physics, 2023, 10(1): 67] proposes to utilize Unet to extract first 30-minute data features to predict full frame images and estimate kinetic parameters. In addition, there are many end-to-end neural networks used to directly reconstruct parameter images, but these methods cannot flexibly arrange time of data acquisition, and most of the methods cannot estimate all parameters at the same time.

### SUMMARY OF INVENTION

In view of the above, the present invention provides a parameter estimation method for compartment model based on physics-informed neural networks, which can estimate kinetic parameter images from a small amount of measurement data from a physical model existing in some medical imaging technologies, and has strong noise robustness and interpretability.

A parameter estimation method for compartment model based on physics-informed neural networks, comprising the following steps:

(1) utilizing the compartment model to obtain measurement data of a tracer in each ROI according to kinetic parameters and an arterial blood input function (AIF) of the tracer in each ROI of a biological tissue;

(2) repeating step (1) to change values of each kinetic parameter and parameters of the arterial blood input function, so as to obtain a large number of samples, wherein each group of samples contains the measurement data of the ROI and a corresponding arterial blood input function;

(3) constructing a physics-informed neural network to solve kinetic parameters of a quantified physiological process in the compartment model; and

(4) utilizing the measurement data of the samples as labels to train the neural network, and extracting corresponding kinetic parameters from network parameters after the training and reconstructing kinetic parameter images.

Further, the compartment model in step (1) satisfies that for any compartment m, a tracer concentration change of a pixel i in the measurement data is expressed as follows:

$$\frac{{dC}_{mi} ( t )}{dt} = {f}_{m} ( {C}_{0 i} ( t ) , {C}_{1 i} ( t ) , {C}_{2 i} ( t ) , … , {C}_{Mi} ( t ) , t , {k}_{1} , {k}_{2} , {k}_{3} , {k}_{4} , … )$$

wherein Cₘᵢ(t) is a concentration of the pixel i at a moment t of the tracer in the compartment m, C₀ᵢ(t) is a concentration of the pixel i at the moment t of the tracer in arterial blood, fₘ( ) represents a linear differential equation of the first order with constant coefficients of the compartment m, m=1,2, . . . . M, m is a serial number of the compartment, M is the number of compartments, k₁, k₂, k₃, k₄, . . . are rate constants, and t represents time.

Further, an expression of the concentration C₀ᵢ(t) is as follows:

$${C}_{0 i} ( t ) = {f}_{0} ( {A}_{1} , {A}_{2} , {A}_{3} , … , t , {λ}_{1} , {λ}_{2} , {λ}_{3} , … )$$

wherein A₁, A₂, A₃, . . . are eigenvalues of an arterial blood input function model, λ₁, λ₂, λ₃, . . . are coefficients of the arterial blood input function model, and f₀( ) is an arterial blood input function of a linear combination of basis functions.

Further, for dynamic measurement of tracer concentration images, at the k^{th} scan, the concentration λᵢₖ of the pixel i in the obtained measurement data is expressed as follows:

$${λ}_{ik} = {f}_{c} ( {C}_{Ti} ( t ) , t , {t}_{k} , {t}_{k - 1} )$$

wherein tₖ represents the current moment, tₖ₋₁ represents the previous moment, C_{Ti}(t) represents the total concentration of the pixel i in the biological tissue observable in a biochemical process at the moment t, and f_{c}( ) is the signal processing function of the measuring instrument on the biological tissue, which is used to process the signal in the tissue into a form of image frame visualization.

Further, the expression of the concentration C_{Ti}(t) is as follows:

$${C}_{Ti} ( t ) = {f}_{T} ( {C}_{0 i} ( t ) , {C}_{1 i} ( t ) , {C}_{2 i} ( t ) , … , {C}_{Mi} ( t ) , t , {V}_{Bi} )$$

wherein V_{Bi} represents the blood vessel volume fraction at the pixel i, and f_{T}( ) is the macroscopic measurable function of the biological tissue, which is in a form of a linear superposition of each compartment and a blood signal.

Further, the physics-informed neural network takes time t as an input and utilizes the quadratic residual network fₓ(t; θ, ω) to calculate the concentration of each pixel at the moment t in each compartment, the network has 6 hidden layers, each hidden layer has 1,024 neurons, and a fifth-order Runge-Kutta is used to improve calculation accuracy; and for all pixels in the image and uniformly discrete time points tⱼ, the neural network approximates the compartment model according to the network parameters θ and kinetic parameters ω so as to predict output measurement data.

Further, the specific process of training the neural network in step (4) is as follows:

4.1 initializing the parameters of the neural network, including the bias vector and the weight matrix of each layer, the learning rate, the maximum number of iterations and the optimizer;

4.2 inputting the uniformly discrete time point tⱼ into the network, obtaining a series of measurement data from an output term of the network through a prediction of a physical equation, calculating the loss function L between the series of measurement data and corresponding measurement data known in the samples, so that the output of the network is constrained by the data item L_{Data}, boundary term L_{B} and the residual term L_{Res} of the ODE; and

4.3 according to the loss function L, utilizing the optimizer to iteratively update the network parameter θ and the kinetic parameter ω by the gradient descent method until the loss function L converges and the training is completed.

Further, the expression of the loss function L is as follows:

$$L = {μ}_{1} ( {L}_{Data} + {L}_{B} ) + {μ}_{2} {L}_{Res}$$

$${L}_{Data} = \frac{1}{N} {\sum }_{n = 1}^{N} {\vert {Λ}_{n} ( θ , ω ) - {Λ}_{n} \vert}^{2}$$

$${L}_{B} = \frac{1}{{N}_{P}} ( {\vert {C}_{1} ( 0 ) - {C}_{1} ( 0 ; θ , ω ) \vert}^{2} + {\vert {C}_{2} ( 0 ) - {C}_{2} ( 0 ; θ , ω ) \vert}^{2} + … + {\vert {C}_{M} ( 0 ) - {C}_{M} ( 0 ; θ , ω ) \vert}^{2} )$$

$${L}_{Res} = \frac{1}{{N}_{P} {N}_{T}} {\sum }_{j = 1}^{{N}_{T}} ( {\vert {Res}_{1 , j} \vert}^{2} + {\vert {Res}_{2 , j} \vert}^{2} + … + {\vert {Res}_{M , j} \vert}^{2} + {\vert {Res}_{T , j} \vert}^{2} )$$

$${Res}_{1 , j} = \frac{\partial {C}_{1} ( {t}_{j} ; θ , ω )}{\partial t} - {f}_{1} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$${Res}_{2 , j} = \frac{\partial {C}_{2} ( {t}_{j} ; θ , ω )}{\partial t} - {f}_{2} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$$…$$

$${Res}_{M , j} = \frac{\partial {C}_{M} ( {t}_{j} ; θ , ω )}{\partial t} - {f}_{M} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$${Res}_{T , j} = \frac{\partial {C}_{T} ( {t}_{j} ; θ , ω )}{\partial t} - \frac{\partial ( {f}_{T} ( {C}_{0} ( t ) , {C}_{1} ( t ) , {C}_{2} ( t ) , … , {C}_{M} ( t ) , t , {V}_{Bi} ) )}{\partial t}$$

wherein Λₙ(θ, ω) is the n^{th} group of measurement data output by the neural network, Λₙ is the measurement data corresponding to the n^{th} group of samples, N is the number of samples, Cₘ(0) represents the concentration image of the tracer in the compartment m at an initial moment in sample measurement data, Cₘ(0; θ, ω) represents the concentration image of the tracer in the compartment m at the initial moment in network output measurement data, Cₘ(t) represents the concentration image of the tracer in the compartment m at moment t in the sample measurement data, C₀(t) represents the concentration image of the tracer in the arterial blood at the moment t, Cₘ(tⱼ; θ, ω) represents the concentration image of the tracer in the compartment m at the moment tⱼ in the network output measurement data, CT (tⱼ; θ, ω) represents the total concentration image of the biological tissue observable in the biochemical process at the moment tⱼ in the network output measurement data, N_{P} is the number of pixels, N_{T} is the number of discrete time points, and μ₁ and μ₂ are the weight coefficients.

The parameter estimation method for compartment model based on physics-informed neural networks of the present invention can extract information from the AIF and a small amount of measurement data to obtain the kinetic parameters, thereby greatly improving the scanning efficiency of measuring instrument, and reducing an inaccurate estimation result due to patient movement. In addition, the present invention has the robustness to AIF noise and measurement data noise, can flexibly arrange the time of data acquisition, reduce the error of inaccurate estimation caused by long time acquisition and the patient movement, and improve the efficiency of data acquisition of the instrument. Experimental results show that the method of the present invention is more stable than the traditional nonlinear least square method in a PET field, and errors in the estimation of some kinetic parameters are obviously smaller than that of the traditional method. Meanwhile, the method of the present invention requires less data, and is superior to the end-to-end supervised reconstruction method U-net network with fewer samples.

### BRIEF DESCRIPTION OF THE DRAWINGS

FIG. 1 is the flow diagram of the parameter estimation method for compartment model based on physics-informed neural networks of the present invention.

FIG. 2 is the structural diagram of a physics-informed neural network PINN framework.

FIG. 3(a) shows estimation error results of various kinetic parameters in different ROI when different levels of blood input function noise are added in a PET experiment.

FIG. 3(b) shows estimation error results of various kinetic parameters in different ROI when different levels of noise are added to measurement data in a PET experiment.

FIG. 4 is the comparison diagram of bias² and Relative Variance indexes of each ROI reconstructed by three methods including the present invention in a PET experiment.

FIG. 5 shows the linear fitting result of all kinetic parameter values and true values under three scanning time schemes in the PET experiment.

### DESCRIPTION OF THE EMBODIMENTS

In order to describe the present invention more clearly, technical schemes of the present invention are described in detail in combination with accompanying drawings and specific embodiments.

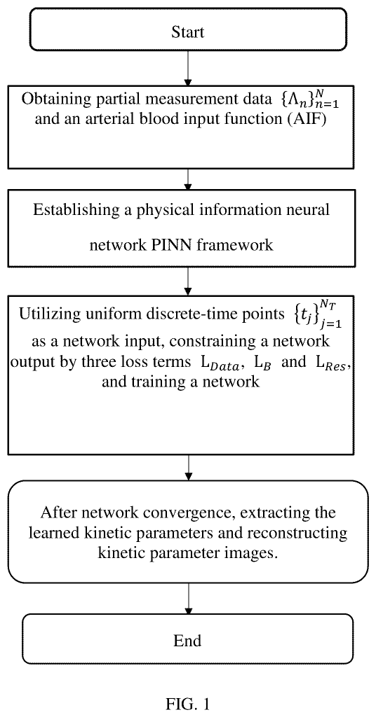

As shown in FIG. 1, the parameter estimation method for compartment model based on physics-informed neural networks of the present invention comprises the following steps:

(1) by utilizing kinetic parameters of the tracer, the drug and other substances involved in a biochemical reaction in each ROI and an arterial blood input function AIF, a compartment model can be used to obtain measurement data Λ of these substances in each ROI; and a large number of samples are obtained by varying the value of each kinetic parameter and parameters of the arterial blood input function to simulate the commonality of different substances, wherein each group of samples contain the measurement data Λ and the arterial blood input function AIF.

The kinetic model principle is as follows: for each compartment m (m=1, . . . ,M), the change of substance concentration in each pixel i (i=1, . . . ,N_{P}) is expressed by a linear first-order constant coefficient ODE equation as:

$$\frac{{dC}_{mi} ( t )}{dt} = {f}_{m} ( {C}_{0 i} ( t ) , {C}_{1 i} ( t ) , {C}_{2 i} ( t ) , … , {C}_{Mi} ( t ) , t , {k}_{1} , {k}_{2} , {k}_{3} , {k}_{4} , … )$$

wherein Cₘᵢ(t) is the concentration of the substance at the moment t in the compartment m, C₀ᵢ(t) is the concentration of the substance at the moment t in arterial blood and can be expressed by the arterial blood input function AIF, and k₁, k₂, k₃, k₄, . . . is rate constants.

Taking a classic two-tissue compartment model (2TCM) as an example, which can be characterized by two ODE equations:

$$[ \begin{matrix}{\dot{C}}_{1 i} ( t ) \\ {\dot{C}}_{2 i} ( t )\end{matrix} ] [ \begin{matrix}- ( {k}_{2} + {k}_{3} ) & {k}_{4} \\ {k}_{3} & - {k}_{4}\end{matrix} ] [ \begin{matrix}{C}_{1 i} ( t ) \\ {C}_{2 i} ( t )\end{matrix} ] + [ \begin{matrix}{k}_{1} \\ 0\end{matrix} ] {C}_{0} ( t )$$

The arterial blood input function model is as follows:

$${C}_{0 i} ( t ) = {f}_{0} ( {A}_{1} , {A}_{2} , {A}_{3} , … , t , {λ}_{1} , {λ}_{2} , {λ}_{3} , … ) = ( {A}_{1} t - {A}_{2} - {A}_{3} ) {e}^{- {λ}_{1} t} + {A}_{2} {e}^{- {λ}_{2} t} + {A}_{3} {e}^{- {λ}_{3} t}$$

wherein λ₁, λ₂, λ₃, . . . A₁, A₂, A₃, . . . are eigenvalues and coefficients of the model respectively.

The total observable concentration C_{Ti}(t) of a biochemical process in the tissue can be expressed as:

$${C}_{Ti} ( t ) = {f}_{T} ( {C}_{0 i} ( t ) , {C}_{1 i} ( t ) , {C}_{2 i} ( t ) , … , {C}_{Mi} ( t ) , t , {V}_{Bi} ) = ( 1 - {V}_{Bi} ) ( {C}_{1 i} ( t ) + {C}_{2 i} ( t ) ) + {V}_{Bi} {C}_{WB} ( t )$$

wherein V_{Bi} is the blood vessel volume fraction at a pixel i.

The measurement model of the measuring instrument is as follows: for dynamic measurement of a substance concentration image, the concentration λᵢₖ of the measured pixel i is obtained at the k^{th} scan:

$${λ}_{ik} = {f}_{c} ( {C}_{Ti} ( t ) , t , {t}_{k} , {t}_{k - 1} ) = \frac{1}{{t}_{k} - {t}_{k - 1}} {\int }_{{t}_{k - 1}}^{{t}_{k}} {C}_{Ti} ( t ) {e}^{- λ t} dt$$

wherein tₖ represents the current moment, tₖ₋₁ represents the previous moment, and finally an image Λₖ=[λ₁ₖ,λ₂ₖ, . . . , λ_{Npk}]^{T} of the k^{th} measurement can be obtained.

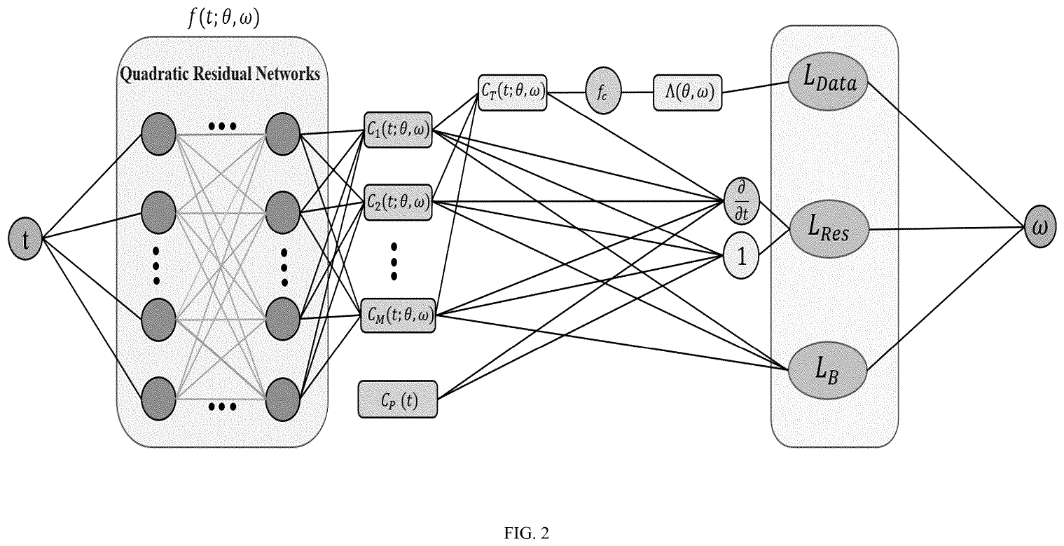

(2) The physics-informed neural network PINN framework is constructed as shown in FIG. 2 to solve the kinetic parameters of a quantified physiological process in the compartment model. Starting from time t, it is input into a quadratic residual network fₓ(t; θ, ω), which can effectively capture more nonlinearity of the model. The network has 6 layers, each hidden layer has 1,024 neurons, and a fifth-order Runge-Kutta is used to improve the calculation accuracy. Assuming that there are M compartments, for all pixels

$${\{ {x}_{i} \}}_{i = 1}^{{N}_{P}}$$

and uniform discrete time points

$${\{ {t}_{j} \}}_{j = 1}^{{N}_{T}}$$

in the image, these equations are approximately solved by using the network parameter j=1 0 and the kinetic parameter ω=[\hat{k}₁, \hat{k}₂, \hat{k}₃, \hat{k}₄, . . . , \hat{V}_{B}], and network outputs C₁ᵢ(t; θ, ω), C₂ᵢ(t; θ, ω), . . . , C_{Mi}(t; θ, ω) etc. are constrained by the data item, the boundary item, and the ODE residual item.

For all pixels in sample measurement data Λ, it is set that C₀=[C₀₁, C₀₂, . . . , C_{0N}_{_{P}}]^{T}, C₁=[C₁₁, C₁₂, . . . , C_{1N}_{_{P}}]^{T}, C₂=[C₂₁, C₂₂, . . . , C_{2N}_{_{P}}]^{T} . . . . C_{M}=[C_{M1}, C_{M2}, . . . , C_{MN}_{_{P}}]^{T}, C_{T}=[C_{T1}, C_{T2}, . . . , C_{TN}_{_{P}}]^{T}.

(3) The PINN network output is constrained by the data item L_{Data}, the boundary item L_{B}, and the ODE residual item L_{Res}.

Assuming that the AIF and part of PET measurement data

$${\{ {Λ}_{n} \}}_{n = 1}^{N}$$

are known, the output of the network receives the data item L_{Data}, the boundary item L_{B}, and the ODE residual item L_{Res} as follows:

$${L}_{Data} = \frac{1}{N} {\sum }_{n = 1}^{N} {\vert {Λ}_{n} ( θ , ω ) - {Λ}_{n} \vert}^{2}$$

$${L}_{B} = \frac{1}{{N}_{P}} ( {\vert {C}_{1} ( 0 ) - {C}_{1} ( 0 ; θ , ω ) \vert}^{2} + {\vert {C}_{2} ( 0 ) - {C}_{2} ( 0 ; θ , ω ) \vert}^{2} + … + {\vert {C}_{M} ( 0 ) - {C}_{M} ( 0 ; θ , ω ) \vert}^{2} )$$

$${Res}_{1 , j} = \frac{\partial {C}_{1} ( {t}_{j} ; θ , ω )}{\partial t} - {f}_{1} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$${Res}_{2 , j} = \frac{\partial {C}_{2} ( {t}_{j} ; θ , ω )}{\partial t} - {f}_{2} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$$…$$

$${Res}_{M , j} = \frac{\partial {C}_{M} ( {t}_{j} ; θ , ω )}{\partial t} - {f}_{M} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$

$${Res}_{T , j} = \frac{\partial {C}_{T} ( {t}_{j} ; θ , ω )}{\partial t} - \frac{\partial ( {f}_{T} ( {C}_{0} ( t ) , {C}_{1} ( t ) , {C}_{2} ( t ) , … , {C}_{M} ( t ) , t , {V}_{Bi} ) )}{\partial t}$$

$${L}_{Res} = \frac{1}{{N}_{P} {N}_{T}} {\sum }_{j = 1}^{{N}_{T}} ( {\vert {Res}_{1 , j} \vert}^{2} + {\vert {Res}_{2 , j} \vert}^{2} + … + {\vert {Res}_{M , j} \vert}^{2} + {\vert {Res}_{T , j} \vert}^{2} )$$

(4) a small amount of measurement data

$${\{ {Λ}_{n} \}}_{n = 1}^{N}$$

is used as labels to train the network, the kinetic parameters are iteratively updated until the network converges, and the training process of the entire network model is as follows:

4.1 initializing the network parameters of the whole model, including the bias vector and the weight matrix of each layer, the learning rate, the maximum number of iterations and the optimizer;

4.2 inputting the uniformly discrete time point

$${\{ {τ}_{j} \}}_{j = 1}^{{N}_{T}}$$

into the network, obtaining a series of predicted measurement data Λₙ(θ, ω) from an output term of the network through a physical equation, calculating the loss L_{Data} with the known part of the measurement data

$${\{ {Λ}_{n} \}}_{n = 1}^{N} ,$$

wherein the total loss of the network is as follows:

$$L = {μ}_{1} ( {L}_{Data} + {L}_{B} ) + {μ}_{2} {L}_{Res}$$

4.3 according to the loss function L, utilizing the optimizer to iteratively update the model parameters θ, ω by the gradient descent method until the loss function converges and the training is completed.

The values of corresponding kinetic parameters are extracted from the learnable parameters of the trained neural network, and the kinetic parameter image is reconstructed.

The following is a simulation experiment based on a two-compartment model in the PET field to verify the effectiveness of the present invention.

A body model used in the experiment is a Zubal brain three-dimensional body model, in which 40 brain slices are taken, and the sample size is 128×128, with two tumors of different sizes. In order to save memory and improve a training speed, we crop an image to 90×90, and total scanning time is 1 hour, which is divided into 27 frames: 2×6 s, 5×12 s, 4×30 s, 4×60 s, 4×120 s and 9×300 s. Uniformly discrete time points N_{T}=1200, an Adam optimizer is used, the learning rate is 5×10⁻⁵, Scal=5, μ₁=2000, and μ₂=1. The model is implemented with pytorch 1.12.0, and all experiments are conducted on a single NVIDIA RTX 3080 GPU, with a total of 20,000 epoches trained.

In the noise robustness experiment, image data of the first 30 minutes (21 frames) are used to estimate kinetic parameters. For AIF and PET activity graph data, their noise e₁ and e₂ meet a Gaussian distribution of a mean value 0 and a variance satisfies the following equation:

$${σ}^{2} ( {e}_{1} ( {t}_{j} ) ) = {( {c}_{1} {C}_{P} ( {t}_{j} ) \cdot {e}^{- λ {t}_{j}} )}^{2}$$

$${σ}^{2} ( {e}_{2} ( {\overline{t}}_{k} ) ) = \frac{{c}_{2} {Λ}_{k}}{{t}_{k} - {t}_{k - 1}} {e}^{- λ {\overline{t}}_{k}}$$

wherein c₁ represents the variation coefficient of C_{P} (which can be assumed to be constant), c₂ is the proportion constant (which determines a level of image noise in a measurement), and t̅ₖ is average frame time; and c1 changes at 0, 2.5%, 5%, 10%, 20%, and c2 changes at 2.5%, 5%, 10%, 20%, 40%.

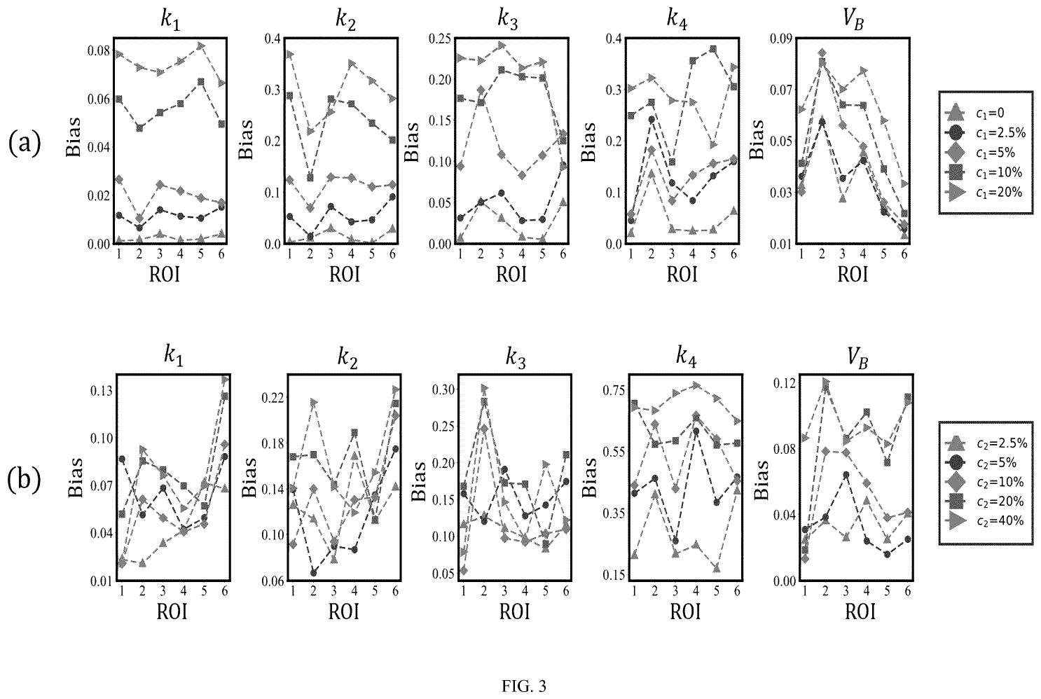

For each coefficient change, ten groups of experiments with different kinetic parameters are tested and an average deviation of each ROI is calculated. FIG. 3(a) shows the result of adding noise to AIF. k₁˜k₃ show noise sensitivity to the AIF. Compared with other kinetic parameters, the network shows the highest estimation accuracy for k₁ and V_{B}, and the lowest estimation accuracy for k₄, yet it has the same anti-noise capability as V_{B}. FIG. 3(b) shows a result of adding noise to PET image data. From closeness of a curve, it can be seen that the network is more robust to the image noise, and the estimation accuracy is still different for different kinetic parameters.

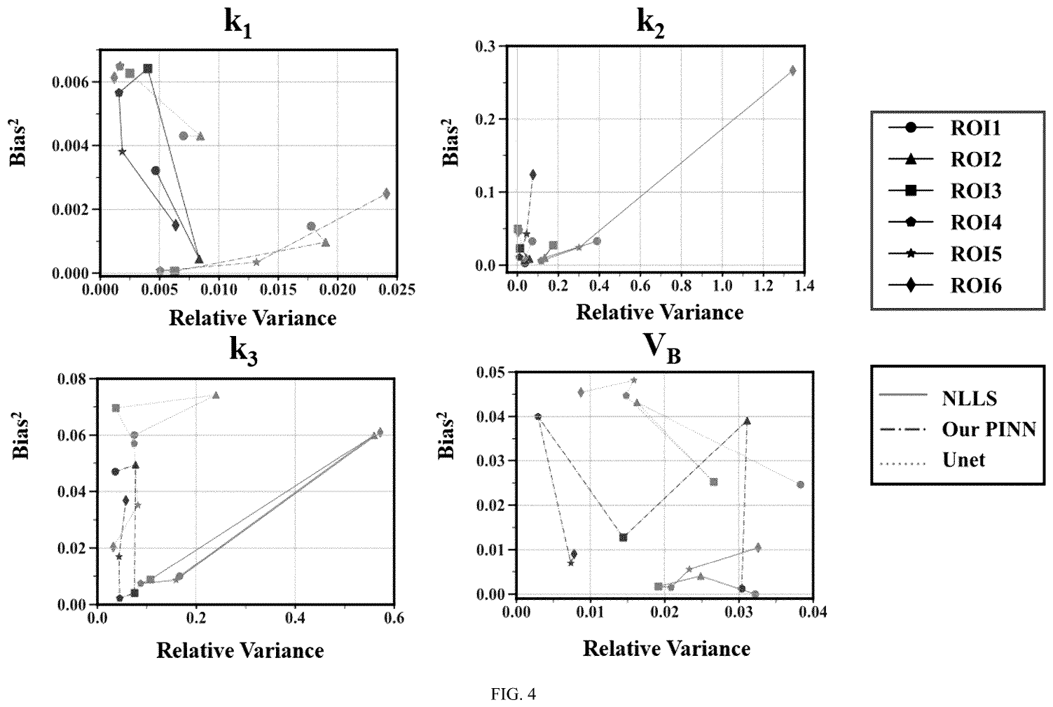

In the comparison experiment, c₁=20% and c₂=40% are set at the same time, and three parameter estimation methods are compared, comprising the NLLS and an end-to-end Unet parameter reconstruction method, each method only use the scan data of the first 30 minutes. For the UNet method, labeled data is used for supervised learning, and the parameter map is reconstructed directly from PET images of the first 30 minutes. 210 PET image samples are used as a training set, each method performs 20 different experiments (with different specific values) at the same level of noise. Relative Variance and bias² are used to measure the experimental result, and the result shows that: the deep learning method generally has stability and repeatability compared to the traditional method, as shown in FIG. 4, especially for a smaller ROI, such as in ROI6 (tumor), and the method of the present invention is significantly superior to the NLLS in estimating k₂ and k₃.

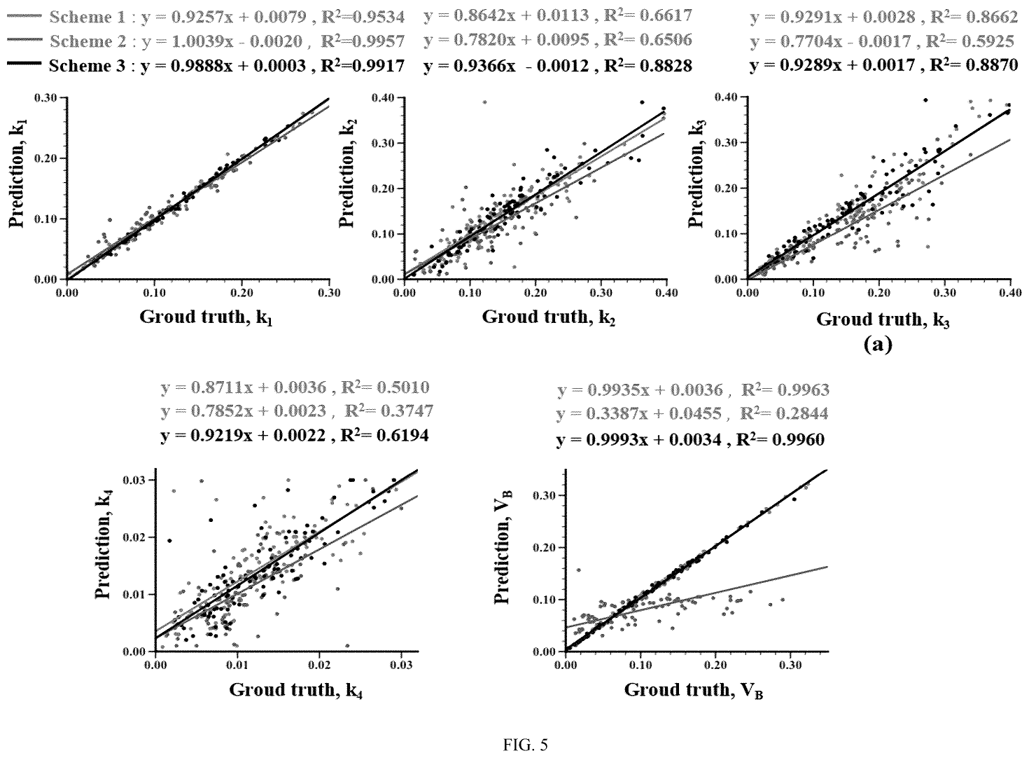

In order to minimize the estimation error, the scanning time of a patient is greatly reduced and flexibly arranged, and thus the efficiency of the PET instrument is improved, three scanning time schemes are explored: (1) the first 30 minutes of scanning (1-21 frames); (2) intermediate 30 minutes of scanning (13-22 frames); and (3) about the first ten minutes (1 to 16 frames) and the last ten minutes (26 to 27 frames) of scanning. As shown in FIG. 5, the linear fitting result between true values and predicted values of the least square method are described. The estimation results of the three schemes for the kinetic parameter k₄ are all poor, and the third scheme requires the least scanning time, but the estimation result is the most accurate. The second scheme has the largest estimation error, indicating that although the noise of the first few frames is large, it has a relatively large impact on the accuracy of the parameter estimation (especially V_{B}).

The above description of embodiments is intended to facilitate understanding and application of the present invention by a person skilled in the art, and obviously, a person familiar with techniques in the art can easily make various modifications to the above embodiments and apply the general principles described herein to other embodiments without creative labor. Therefore, the present invention is not limited to the above embodiments, and an improvement and modification of the present invention made by a person skilled in the field according to the disclosure of the present invention shall be within a protection scope of the present invention.

What is claimed is:

1. A parameter estimation method for compartment model based on physics-informed neural networks, comprising the following steps:
(1) utilizing the compartment model to obtain measurement data of a tracer in each ROI according to kinetic parameters and an arterial blood input function of the tracer in each ROI of a biological tissue;
(2) repeating step (1) to change values of each kinetic parameter and a parameter of the arterial blood input function, so as to obtain a large number of samples, wherein each group of samples contains the measurement data of the ROI and the corresponding arterial blood input function;
(3) constructing a physics-informed neural network to solve kinetic parameters of a quantified physiological process in the compartment model; and
(4) utilizing the measurement data of the samples as labels to train the neural network, and extracting corresponding kinetic parameters from network parameters after the training and reconstructing kinetic parameter images.

2. The parameter estimation method for compartment model based on physics-informed neural networks according to claim 1, wherein the compartment model in step (1) satisfies that for any compartment m, a tracer concentration change of a pixel i in the measurement data is expressed as follows:
$$\frac{{dC}_{mi} ( t )}{dt} = {f}_{m} ( {C}_{0 i} ( t ) , {C}_{1 i} ( t ) , {C}_{2 i} ( t ) , … , {C}_{Mi} ( t ) , t , {k}_{1} , {k}_{2} , {k}_{3} , {k}_{4} , … )$$
wherein Cₘᵢ(t) is a concentration of the pixel i at a moment t of the tracer in the compartment m, C_{Oi}(t) is a concentration of the pixel i at the moment t of the tracer in arterial blood, fₘ( ) represents a linear differential equation of the first order with constant coefficients of the compartment m, m=1,2, . . . . M, m is a serial number of the compartment, M is the number of compartments, k₁, k₂, k₃, k₄, . . . are rate constants, and t represents time.

3. The parameter estimation method for compartment model based on physics-informed neural networks according to claim 2, wherein the expression of the concentration C₀ᵢ(t) is as follows:
$${C}_{0 i} ( t ) = {f}_{0} ( {A}_{1} , {A}_{2} , {A}_{3} , … , t , {λ}_{1} , {λ}_{2} , {λ}_{3} , … )$$
wherein A₁, A₂, A₃, . . . are eigenvalues of the arterial blood input function model, λ₁, λ₂, λ₃, . . . are coefficients of the arterial blood input function model, and f₀( ) is the arterial blood input function of a linear combination of basis functions.

4. The parameter estimation method for compartment model based on physics-informed neural networks according to claim 3, wherein for kinetic measurement of the tracer concentration image, at the k^{th} scan, the concentration λᵢₖ of the pixel i in the obtained measurement data is expressed as follows:
$${λ}_{ik} = {f}_{c} ( {C}_{Ti} ( t ) , t , {t}_{k} , {t}_{k - 1} )$$
wherein tₖ represents the current moment, tₖ₋₁ represents the previous moment, C_{Ti}(t) represents the total concentration of the pixel i in the biological tissue observable in a biochemical process at the moment t, and f_{c}( ) is a signal processing function of a measuring instrument on the biological tissue, which is used to process a signal in the tissue into a form of image frame visualization.

5. The parameter estimation method for compartment model based on physics-informed neural networks according to claim 4, wherein the expression of the total concentration C_{Ti}(t) is as follows:
$${C}_{Ti} ( t ) = {f}_{T} ( {C}_{0 i} ( t ) , {C}_{1 i} ( t ) , {C}_{2 i} ( t ) , … , {C}_{Mi} ( t ) , t , {V}_{Bi} )$$
wherein V_{Bi} represents the blood vessel volume fraction at the pixel i, and f_{T}( ) is a macroscopic measurable function of the biological tissue, which is in a form of a linear superposition of each compartment and a blood signal.

6. The parameter estimation method for compartment model based on physics-informed neural networks according to claim 1, wherein the physics-informed neural networks takes time t as an input and utilizes a quadratic residual network fₓ(t; θ, ω) to calculate a concentration of each pixel at the moment t in each compartment, the network has 6 hidden layers, each hidden layer has 1,024 neurons, and a fifth-order Runge-Kutta is used to improve the calculation accuracy; and for all pixels in the image and a uniformly discrete time point tⱼ, the neural network approximates the compartment model according to the network parameters θ and kinetic parameters ω so as to predict output measurement data.

7. The parameter estimation method for compartment model based on physics-informed neural networks according to claim 5, wherein a specific process of training the neural network in step (4) is as follows:
4.1 initializing the parameters of the neural network, including the bias vector and the weight matrix of each layer, the learning rate, the maximum number of iterations and an optimizer;
4.2 inputting the uniformly discrete time point tⱼ into the network, obtaining a series of measurement data from an output term of the network through a prediction of a physical equation, calculating the loss function L between the series of measurement data and corresponding measurement data known in the samples, so that an output of the network is constrained by the data item L_{Data}, the boundary term L_{B} and the residual term L_{Res} of an ODE; and
4.3 according to the loss function L, utilizing the optimizer to iteratively update the network parameter θ and the kinetic parameter ω by the gradient descent method until the loss function L converges and the training is completed.

8. The parameter estimation method for compartment model based on physics-informed neural networks according to claim 7, wherein the expression of the loss function Lis as follows:
$$L = {μ}_{1} ( {L}_{Data} + {L}_{B} ) + {μ}_{2} {L}_{Res}$$
$${L}_{Data} = \frac{1}{N} {\sum }_{n = 1}^{N} {\vert {Λ}_{n} ( θ , ω ) - {Λ}_{n} \vert}^{2}$$
$${L}_{B} = \frac{1}{{N}_{P}} ( {\vert {C}_{1} ( 0 ) - {C}_{1} ( 0 ; θ , ω ) \vert}^{2} + {\vert {C}_{2} ( 0 ) - {C}_{2} ( 0 ; θ , ω ) \vert}^{2} + … + {\vert {C}_{M} ( 0 ) - {C}_{M} ( 0 ; θ , ω ) \vert}^{2} )$$
$${L}_{Res} = \frac{1}{{N}_{P} {N}_{T}} {\sum }_{j = 1}^{{N}_{T}} ( {\vert {Res}_{1 , j} \vert}^{2} + {\vert {Res}_{2 , j} \vert}^{2} + … + {\vert {Res}_{M , j} \vert}^{2} + {\vert {Res}_{T , j} \vert}^{2} )$$
$${Res}_{1 , j} = \frac{\partial {C}_{1} ( {t}_{j} ; θ , ω )}{\partial t} - {f}_{1} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$
$${Res}_{2 , j} = \frac{\partial {C}_{2} ( {t}_{j} ; θ , ω )}{\partial t} - {f}_{2} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$
$$…$$
$${Res}_{M , j} = \frac{\partial {C}_{M} ( {t}_{j} ; θ , ω )}{\partial t} - {f}_{M} ( {C}_{0} ( t ) , {C}_{1} ( t ) , {C}_{2} ( t ) , … , {C}_{M} ( t ) , t , {k}_{1} , {k}_{2} , {k}_{3} , {k}_{4} , … )$$
$${Res}_{T , j} = \frac{\partial {C}_{T} ( {t}_{j} ; θ , ω )}{\partial t} - \frac{\partial ( {f}_{T} ( {C}_{0} ( t ) , {C}_{1} ( t ) , {C}_{2} ( t ) , … , {C}_{M} ( t ) , t , {V}_{Bi} ) )}{\partial t}$$
wherein Λₙ(θ, ω) is a n^{th} group of measurement data output by the neural network, Λₙ is the measurement data corresponding to the n^{th} group of samples, Nis the number of samples, Cₘ(0) represents the concentration image of the tracer in the compartment m at an initial moment in sample measurement data, Cₘ(0; θ, ω) represents the concentration image of the tracer in the compartment m at the initial moment in network output measurement data, Cₘ(t) represents the concentration image of the tracer in the compartment m at moment t in the sample measurement data, C₀(t) represents the concentration image of the tracer in the arterial blood at the moment t, Cₘ(tⱼ; θ, ω) represents the concentration image of the tracer in the compartment m at the moment tⱼ in the network output measurement data, C_{T}(tⱼ; θ, ω) represents the total concentration image of the biological tissue observable in the biochemical process at the moment tⱼ in the network output measurement data, N_{P} is the number of pixels, N_{T} is the number of discrete time points, and μ₁ and μ₂ are the weight coefficients.
